(19) United States
(12) Patent Application Publication
(10) Pub. No.: US 2017/0366879 A1
(43) Pub. Date: Dec. 21, 2017

(54) PLANT MONITOR, METHOD AND APPARATUS FOR GENERATING INFORMATION

(71) Applicant: Beijing Xiaomi Mobile Software Co., Ltd., Beijing (CN)

(72) Inventors: Qiang FU, Beijing (CN); Shiquan ZHOU, Beijing (CN); Enxing HOU, Beijing (CN)

(21) Appl. No.: 15/623,676

(22) Filed: Jun. 15, 2017

(30) Foreign Application Priority Data
Jun. 17, 2016 (CN) 201680000727.7

(51) Int. Cl. H04Q 9/02; G01J 1/18; G01K 13/00; G09B 5/02; A01G 9/00; G01N 27/04; G01N 33/24; G09B 19/00

(52) U.S. Cl. CPC H04Q 9/02; G01N 33/24; G01J 1/18; G09B 19/00; G09B 5/02; A01G 9/00; G01N 27/04; G01K 13/00; H04Q 2209/40; H04Q 2209/82

(57) ABSTRACT

A plant monitor includes: a main control chip, a wireless communication chip connected with the main control chip, and at least one growth environment monitoring sensor connected with the main control chip. The at least one growth environment monitoring sensor includes at least one of an illumination sensor, a temperature sensor, a humidity sensor, or a soil conductivity sensor. The main control chip is configured to acquire growth environment data collected by the at least one growth environment monitoring sensor, and send the growth environment data to a mobile terminal via the wireless communication chip. The mobile terminal is configured to generate plant cultivation reference information according to the growth environment data.

### CROSS-REFERENCE TO RELATED APPLICATIONS

This application is based upon and claims priority to Chinese Patent Application No. 201680000727.7 filed on Jun. 17, 2016, the entire contents of which are incorporated herein by reference.

### TECHNICAL FIELD

The present disclosure generally relates to a field of electronic devices, and more particularly, to a plant monitor, a method and an apparatus for generating information, and a system for monitoring a plant.

### BACKGROUND

Plant cultivation, such as ornamental plant cultivation, is one of common hobbies in daily life. The ornamental plant can purify air, relax nerves, and promote health.

In a process of cultivating the ornamental plant, a user needs to perform lighting, watering, and fertilization on the ornamental plant in accordance with living habits of the ornamental plant. However, it is difficult for the user to cultivate the ornamental plant to a better state without knowing the living habits, because ornamental plants are becoming more and more diversified.

### SUMMARY

According to a first aspect of the present disclosure, a plant monitor is provided. The plant monitor includes: a main control chip, a wireless communication chip connected with the main control chip, and at least one growth environment monitoring sensor connected with the main control chip. The at least one growth environment monitoring sensor includes at least one of an illumination sensor, a temperature sensor, a humidity sensor, or a soil conductivity sensor. The main control chip is configured to acquire growth environment data collected by the at least one growth environment monitoring sensor, and send the growth environment data to a mobile terminal via the wireless communication chip, such that the mobile terminal generates plant cultivation reference information according to the growth environment data.

According to a second aspect of the present disclosure, a method for generating information is provided. The method includes: receiving growth environment data sent by a plant monitor, wherein the growth environment data is collected by the plant monitor via a growth environment monitoring sensor, and the growth environment monitoring sensor includes at least one of an illumination sensor, a temperature sensor, a humidity sensor, or a soil conductivity sensor; and generating plant cultivation reference information according to the growth environment data.

According to a third aspect of the present disclosure, an apparatus for generating information is provided. The apparatus includes: a processor; and a memory configured to store an instruction executable by the processor. The processor is configured to: receive growth environment data sent by a plant monitor, wherein the growth environment data is collected by the plant monitor via a growth environment monitoring sensor, and the growth environment monitoring sensor includes at least one of an illumination sensor, a temperature sensor, a humidity sensor, or a soil conductivity sensor; and generate plant cultivation reference information according to the growth environment data.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments consistent with the present disclosure and, together with the description, serve to explain the principles of the present disclosure.

FIG. 1 is a schematic diagram illustrating a system for monitoring a plant according to an exemplary embodiment of the present disclosure.

FIG. 2A is a block diagram illustrating a plant monitor according to an exemplary embodiment of the present disclosure.

FIG. 2B is a block diagram illustrating a plant monitor according to another exemplary embodiment of the present disclosure.

FIG. 3 is a flow chart showing a method for generating information according to an exemplary embodiment of the present disclosure.

FIG. 4 is a flow chart showing a method for generating information according to another exemplary embodiment of the present disclosure.

FIG. 5A is a schematic diagram illustrating an interface of a method for generating information according to an exemplary embodiment of the present disclosure.

FIG. 5B is a schematic diagram illustrating an interface of a method for generating information according to an exemplary embodiment of the present disclosure.

FIG. 6 is a flow chart showing a method for generating information according to another exemplary embodiment of the present disclosure.

FIG. 7 is a flow chart showing a method for generating information according to another exemplary embodiment of the present disclosure.

FIG. 8 is a flow chart showing a method for generating information according to another exemplary embodiment of the present disclosure.

FIG. 9 is a flow chart showing a method for generating information according to another exemplary embodiment of the present disclosure.

FIG. 10 is a flow chart showing a method for generating information according to another exemplary embodiment of the present disclosure.

FIG. 11A is a block diagram illustrating an apparatus for generating information according to an exemplary embodiment of the present disclosure.

FIG. 11B is a block diagram illustrating an apparatus for generating information according to another exemplary embodiment of the present disclosure.

FIG. 12 is a block diagram illustrating a mobile terminal according to an exemplary embodiment of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the present disclosure. Instead, they are merely examples of devices and methods consistent with aspects related to the present disclosure as recited in the appended claims.

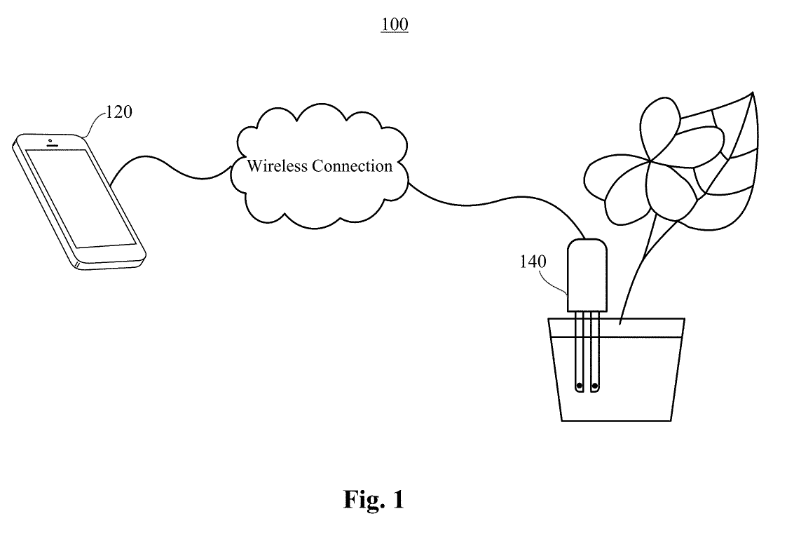

FIG. 1 is a schematic diagram illustrating a system 100 for monitoring a plant according to an exemplary embodiment of the present disclosure. The system 100 includes a mobile terminal 120 and a plant monitor 140.

The mobile terminal 120 can be a mobile phone, a tablet computer, an e-book reader, an MP3 (Moving Picture Experts Group Audio Layer III) player, an MP4 (Moving Picture Experts Group Audio Layer IV) player, a laptop computer, a desktop computer and the like. In one embodiment, a plant-cultivation-type application is installed in the mobile terminal 120.

The mobile terminal 120 is connected to the plant monitor 140 via a wireless connection. The wireless connection can be at least one of a Zigbee connection, a wireless networking standard Z-Wave connection, a Wi-Fi (Wireless Fidelity) connection, a BLE (Bluetooth Low Energy) connection, or an RF (Radio Frequency) connection.

The plant monitor 140 is an instrument or an apparatus for monitoring growth environment data of the plant and for sending the monitored growth environment data to the mobile terminal 120.

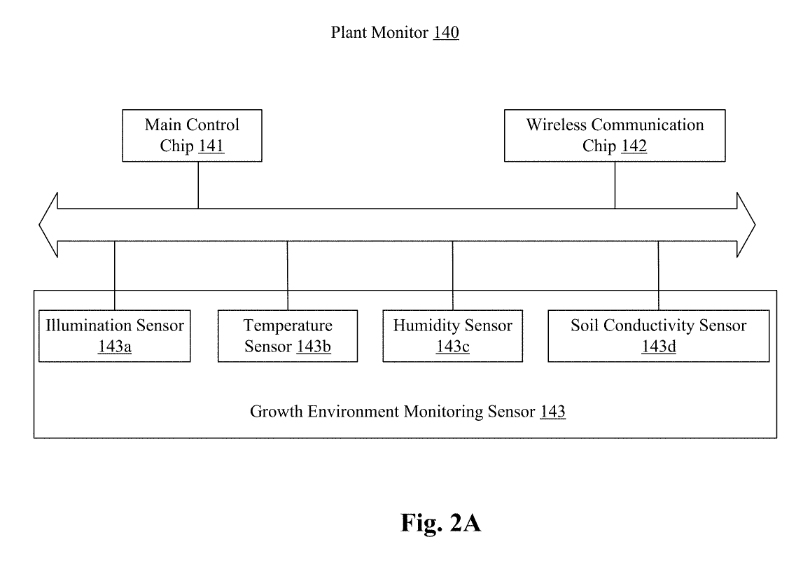

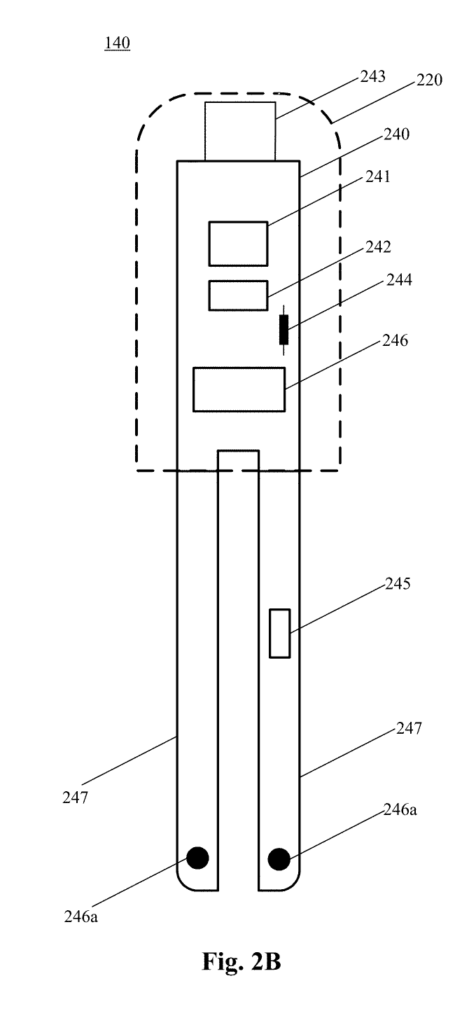

FIG. 2A is a block diagram illustrating the plant monitor 140 (FIG. 1) according to an exemplary embodiment of the present disclosure. The plant monitor 140 includes a main control chip 141, a wireless communication chip 142 connected with the main control chip 141, and at least one growth environment monitoring sensor 143 connected with the main control chip 141.

The at least one growth environment monitoring sensor 143 includes at least one of an illumination sensor 143a, a temperature sensor 143b, a humidity sensor 143c, or a soil conductivity sensor 143d.

The wireless communication chip 142 can be at least one of a Zigbee chip, a Z-Wave chip, a Wi-Fi chip, a BLE chip or an RF chip.

The illumination sensor 143a is configured to collect illumination data of the plant growth environment. The temperature sensor 143b is configured to collect temperature data of the plant growth environment. The humidity sensor 143c is configured to collect air humidity data and/or soil humidity data in the plant growth environment. The soil conductivity sensor 143d is configured to collect electrical conductivity in the plant growth environment. In some embodiments, the electrical conductivity is converted into a soil PH value, or the conductivity is converted into a soil fertility index.

The main control chip 141 is configured to acquire growth environment data collected by the growth environment monitoring sensor 143, and to send the growth environment data to the mobile terminal 120 via the wireless communication chip 142. The mobile terminal 120 (FIG. 1) is configured to generate plant cultivation reference information according to the growth environment data.

In one embodiment, the growth environment data includes at least one of the illumination data, the temperature data, the soil humidity data, or the PH value.

In one embodiment, the main control chip 141 sends the growth environment data to the mobile terminal 120 based on a predetermined transmission strategy. The predetermined transmission strategy includes at least one of sending at every predetermined time interval, sending when the data changes, and sending when receiving a data request sent by the mobile terminal 120.

In one embodiment, the main control chip 141 is further configured to build a binding relationship with the mobile terminal 120 via the wireless communication chip 142.

In an illustrative embodiment, as shown in FIG. 2B, the plant monitor 140 includes a housing 220 and a PCB (printed circuit board) 240 in the housing 220. The PCB 240 is provided with a main control chip 241, a Bluetooth communication chip 242, an illumination sensor 243, a temperature sensor 244, a humidity sensor 245, and a soil conductivity sensor 246. The PCB 240 also includes conductive paths connecting respective components and peripheral circuitry (not shown).

In one embodiment, a region of the housing 220 corresponding to a lighting component of the illumination sensor 243 is transparent. Alternatively, the lighting component of the illumination sensor 243 is exposed on the housing 220. The illumination sensor 243 can be used to measure a maximum light intensity of 100,000 Lux, with an accuracy of 100 Lux.

In one embodiment, the temperature sensor 244 is a thermistor disposed on the PCB 240. The temperature sensor 244 can be used to measure a temperature range from −20 degree Centigrade to 50 degree Centigrade, with an accuracy of ±0.5 degree Centigrade.

In one embodiment, the plant monitor 140 includes an insertion structure configured to be inserted into soil. The insertion structure includes a portion of the PCB 240 extending out of the housing 220. In one embodiment, as illustrated in FIG. 2B, the insertion structure includes two insertion arms 247 disposed in parallel, and each of the insertion arms 247 may be a plate. In an alternative embodiment, the insertion structure includes a single plate to be inserted into the soil. Since the PCB 240 has a corrosion-resistant characteristic, the insertion structure has the corrosion-resistant characteristic after being inserted into the soil for a long period, as compared to a metal insertion arm.

In one embodiment, the humidity sensor 245 is disposed on the insertion arm 247. The humidity sensor 245 is configured to collect soil humidity.

In one embodiment, the soil conductivity sensor 246 includes two metal electrodes 246a. Each one of the two metal electrodes 246a is disposed on one of the insertion arms 247. When two insertions arms 247 disposed in parallel are provided, each insertion arm 247 is provided with one metal electrode 246a. In an alternative embodiment, when the insertion structure includes the single plate, the two metal electrodes 246a are disposed on the single plate.

As will be appreciated by those skilled in the art, the plant monitor 140 also includes more or fewer components than the illustrations above. For example, the plant monitor 140 can also include a power source for supplying power to various components of the plant monitor 140. The power supply can be a coin cell battery or a rechargeable battery.

With the plant monitor 140 provided in the present embodiment, the growth environment data of the plant is collected by the at least one growth environment monitoring sensor 143, such that the mobile terminal 120 can generate the plant cultivation reference information according to the growth environment data. Therefore, the problem that it is difficult for the user to cultivate the plant to a better state without knowing the plant's living habits can be solved. Moreover, the user only needs to insert the plant monitor 140 into the plant growth soil, to acquire the plant cultivation reference information, without having to know the plant's living habits or having to determine appropriateness of the plant growth environment manually.

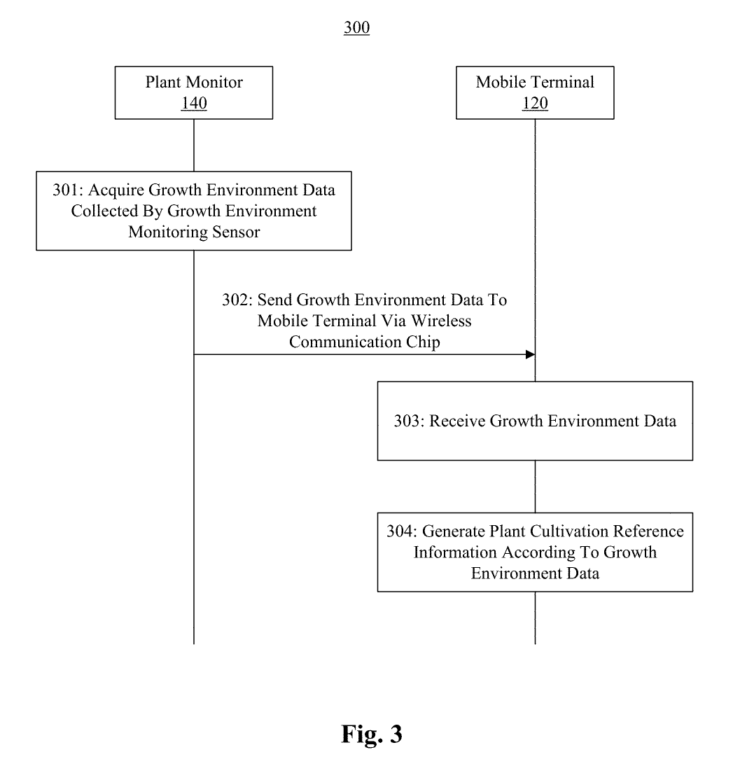

FIG. 3 is a flow chart showing a method 300 for generating information according to an exemplary embodiment of the present disclosure. The present embodiment is exemplified by applying the method 300 to the system 100 for monitoring a plant shown in FIG. 1. The method 300 includes following steps.

In step 301, the plant monitor 140 acquires the growth environment data collected by the at least one growth environment monitoring sensor 143.

In one embodiment, the growth environment data includes at least one of the illumination data, the temperature data, the soil humidity data, or the PH value.

In step 302, the plant monitor 140 sends the growth environment data to the mobile terminal 120 via the wireless communication chip 142.

In one embodiment, the plant monitor 140 sends the growth environment data to the mobile terminal 120 via the wireless communication chip 142 at every predetermined time interval. In one embodiment, when monitoring that the growth environment data changes, the plant monitor 140 sends the growth environment data to the mobile terminal 120 via the wireless communication chip 142. In one embodiment, when receiving a data request sent by the mobile terminal 120, the plant monitor 140 sends the growth environment data to the mobile terminal 120 via the wireless communication chip 142.

In step 303, the mobile terminal 120 receives the growth environment data sent by the plant monitor 140.

In step 304, the mobile terminal 120 generates the plant cultivation reference information according to the growth environment data.

In some embodiments, the plant cultivation reference information includes: information configured to recommend enhancing illumination, information configured to inform the user that illumination is appropriate, information configured to recommend reducing illumination, information configured to recommend enhancing temperature, information configured to inform the user that the temperature is appropriate, information configured to recommend reducing temperature, information configured to recommend watering the plant, information configured to inform the user that the quantity of water is proper, information configured to recommend ventilation, information configured to recommend applying an alkaline fertilizer, information configured to inform the user that the fertilizer is proper, information configured to recommend applying an acidic fertilizer, information configured to recommend applying a first elemental fertilizer, and information configured to recommend applying a second elemental fertilizer.

The first elemental fertilizer has an absorption rate lower than a predetermined condition in acidic soil. The second elemental fertilizer has an absorption rate lower than a predetermined condition in alkaline soil.

With the method 300 for generating information provided in the present embodiment, the growth environment data of the plant is collected by the at least one growth environment monitoring sensor 143 in the plant monitor 140, and the growth environment data is sent to the mobile terminal 120 via the wire communication chip 142, such that the mobile terminal 120 generates the plant cultivation reference information according to the growth environment data. Therefore, the problem that it is difficult for the user to cultivate the plant in a better state without knowing the plant's living habits can be solved. Moreover, the user only needs to insert the plant monitor 140 into the soil to acquire the plant cultivation reference information, without having to know the plant's living habits or having to determine appropriateness of the plant growth environment manually.

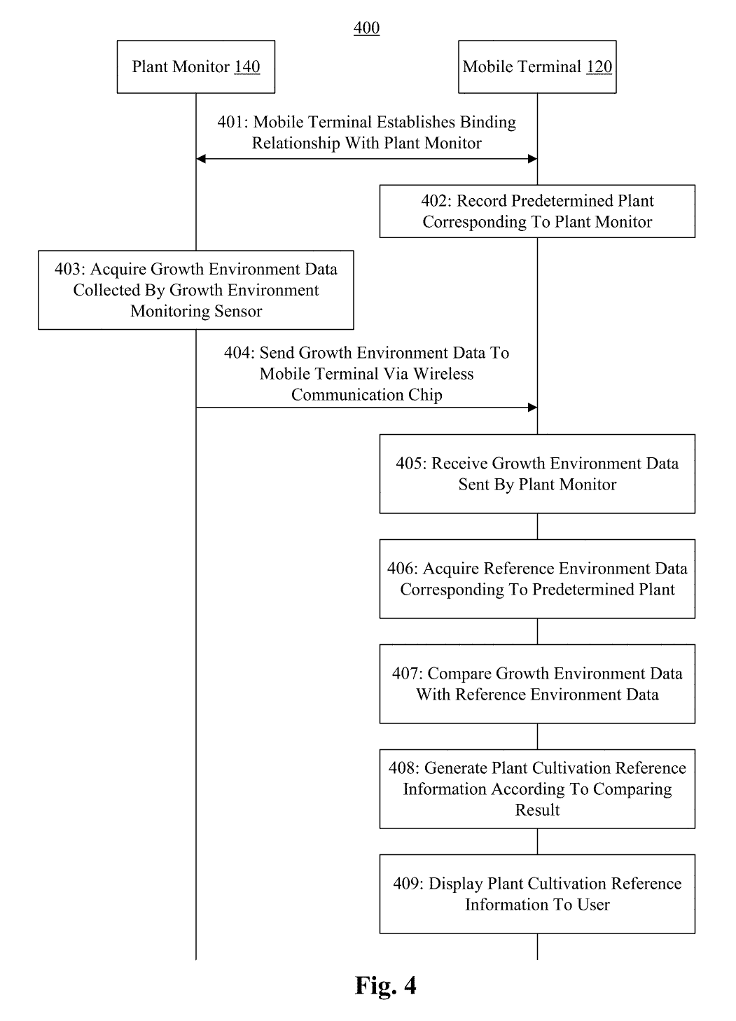

FIG. 4 is a flow chart showing a method 400 for generating information according to an exemplary embodiment of the present disclosure. The present embodiment is exemplified by applying the method 400 for generating information to the system 100 for monitoring a plant shown in FIG. 1. The method 400 includes followings.

In step 401, the mobile terminal 120 establishes a binding relationship with the plant monitor 140.

In one embodiment, the mobile terminal 120 established the binding relationship with the plant monitor 140 as follows.

First, the plant monitor 140 sends a Bluetooth broadcasting message after an initial starting up.

Second, the mobile terminal 120 receives the Bluetooth broadcasting message, and acquires an identifier of the plant monitor 140 according to the Bluetooth broadcasting message.

Third, the mobile terminal 120 binds the identifier of the plant monitor 140 with itself.

After binding, the mobile terminal 120 receives the growth environment data reported only by the bound plant monitor 140.

The embodiment of the present disclosure does not limit specific forms of the binding process.

In step 402, the mobile terminal 120 records a predetermined plant corresponding to the plant monitor 140.

In one embodiment, in the binding process, the mobile terminal 120 displays an information input interface for requesting the user to input the predetermined plant corresponding to the plant monitor 140. The predetermined plant is a plant in the growth environment monitored by the plant monitor 140.

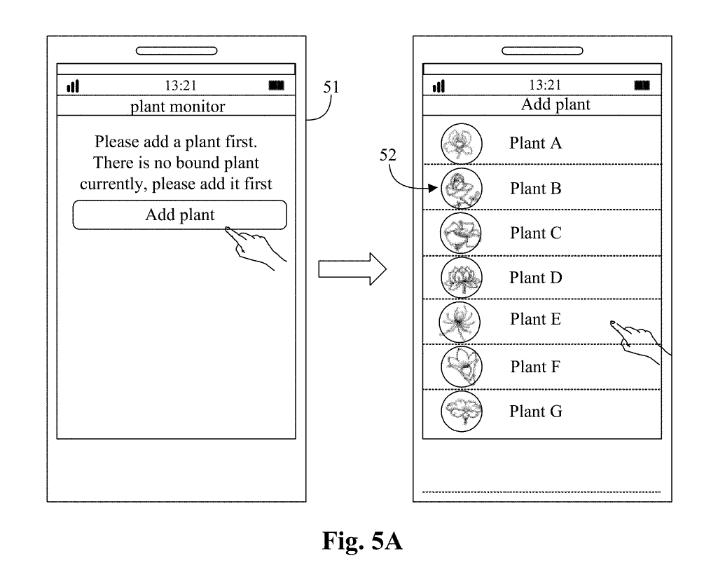

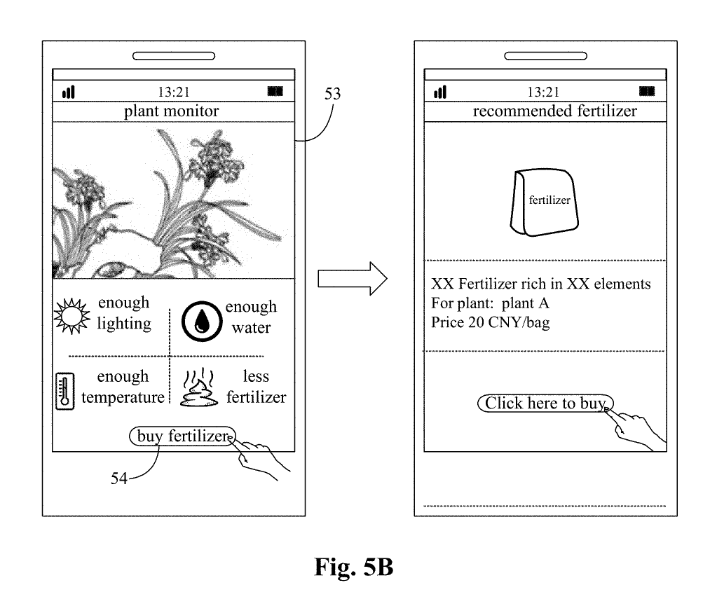

As shown in FIG. 5A, after binding with the plant monitor, a mobile terminal 51 displays an information input interface including a plant selection item 52 for the user to input the predetermined plant corresponding to the plant monitor.

In step 403, the plant monitor 140 acquires the growth environment data collected by the at least one growth environment monitoring sensor 143.

In one embodiment, the growth environment data includes at least one of the illumination data, the temperature data, the soil humidity data, or the PH value.

In step 404, the plant monitor 140 sends the growth environment data to the mobile terminal 120 via the wireless communication chip 142.

In one embodiment, the plant monitor 140 sends the growth environment data to the mobile terminal 120 via the wireless communication chip 142 every predetermined time interval. The predetermined time interval can be 15 minutes, 30 minutes, 1 hour, 2 hours, 4 hours, etc.

In one embodiment, when monitoring that the growth environment data changes, the plant monitor 140 sends the growth environment data to the mobile terminal 120 via the wireless communication chip 142.

In one embodiment, when receiving a data request sent by the mobile terminal 120, the plant monitor 140 sends the growth environment data to the mobile terminal 120 via the wireless communication chip.

In step 405, the mobile terminal 120 receives the growth environment data sent by the plant monitor 140.

In one embodiment, the mobile terminal 120 receives the growth environment data sent by the plant monitor 140 via the wire communication chip 142.

In step 406, the mobile terminal 120 acquires reference environment data corresponding to the predetermined plant.

In one embodiment, the predetermined plant is a plant previously set by the user. The predetermined plant is a plant cultivated in the plant growth environment monitored by the plant monitor.

In one embodiment, different plants correspond to different reference environmental data. Each plant has various reference environmental data at various growth stages.

In one embodiment, the reference environmental data corresponding to the predetermined plant includes at least one of the reference illumination data, the reference temperature data, the reference air humidity data, the reference soil humidity data, or the reference soil pH value.

In step 407, the mobile terminal 120 compares the growth environment data with the reference environment data.

The mobile terminal 120 compares the collected growth environment data, a statistic value of the growth environment data, or a conversion value of the growth environment data, with the reference environment data.

In one embodiment, when the growth environment data includes the illumination data, the illumination data or a statistic value of the illumination data includes at least one of: a maximum light intensity, an average light intensity, a minimum light intensity, a daily lighting duration, a daily average light intensity, a daily average lighting duration, a weekly average light intensity, a weekly average lighting duration, a monthly average light intensity, or a monthly average lighting duration.

In one embodiment, when the growth environment data includes the temperature data, the temperature data or a statistic value of the temperature data includes at least one of: a maximum temperature, an average temperature, a minimum temperature, a daytime average temperature, a nighttime average temperature, a weekly average temperature, or a monthly average temperature.

In one embodiment, when the growth environment data includes the soil humidity data, the soil humidity data or a statistic value of the soil humidity data includes at least one of: a maximum soil humidity, an average soil humidity, a minimum soil humidity, a daytime average soil humidity, a nighttime average soil humidity, a weekly average soil humidity, or a monthly average soil humidity.

In one embodiment, when the growth environment data includes the soil conductivity, the soil conductivity or a statistic value of the soil conductivity or a conversion value of the soil conductivity includes at least one of: a soil salinity, a soil PH value, a soil fertility, a daily average soil salinity, a daily average soil PH value, a daily average soil fertility, a weekly average soil salinity, a weekly average soil PH value, a weekly average soil fertility, a monthly average soil salinity, a monthly average soil PH value, or a monthly average soil fertility.

There are conversion relationships between the soil conductivity and the soil salinity, between the soil conductivity and the soil PH value, and between the soil conductivity and the soil fertility. These conversion relationships can be denoted by functions, curves, data tables, and other forms.

The mobile terminal 120 can compare the various data in the growth environment data with the corresponding data item in the reference environment data.

In step 408, the mobile terminal 120 generates plant cultivation reference information according to a comparing result.

In step 409, the mobile terminal 120 displays the plant cultivation reference information to the user.

The mobile terminal 120 displays the plant cultivation reference information in the plant-cultivation-type application. For example, the plant cultivation reference information includes: adequate light, adequate water, adequate temperature, less fertility, and other reference information.

In one embodiment, referring to FIG. 5B, the mobile terminal simultaneously displays the plant cultivation reference information and a guidance entrance 54 on a graphical user interface 53. The guidance entrance 54 includes at least one of an entrance for buying a cultivation implement, an entrance for buying fertilizer, an entrance for jumping to a cultivation forum, or an entrance for jumping to a cultivation technique.

With the method 400 for generating information provided in the present embodiment, the mobile terminal 120 can compare the growth environment data of the plant with the reference environment data corresponding to the plant and generate the plant cultivation reference information based on the comparison result. Therefore, for each plant, the corresponding plant cultivation reference information can be generated with high reference value.

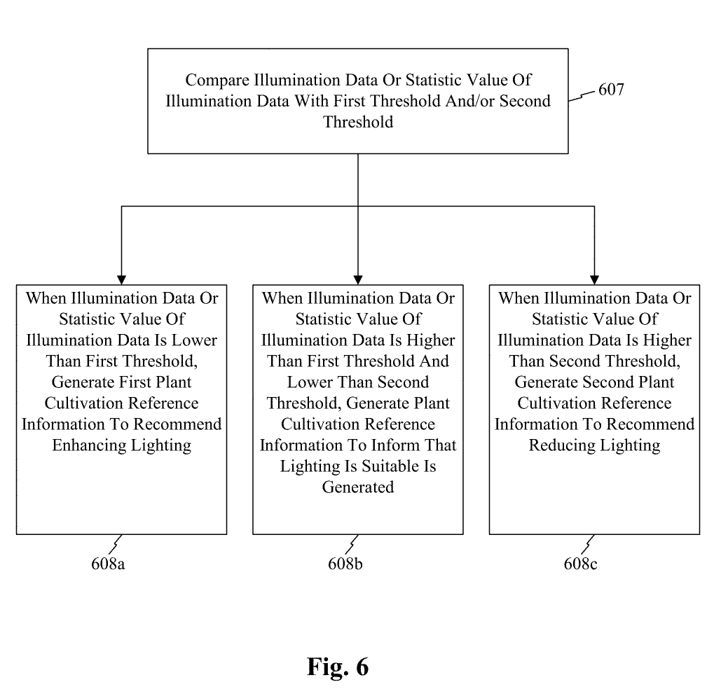

In one embodiment, the growth environment data includes illumination data, and the reference environment data includes a first threshold and a second threshold, in which the first threshold is smaller than the second threshold. Accordingly, the following steps in FIG. 6, are performed to implement steps 407 and 408 in FIG. 4.

In step 607, the illumination data or a statistic value of the illumination data is compared with the first threshold, and/or the illumination data or the statistic value of the illumination data is compared with the second threshold.

For example, the illumination data includes the daily average lighting duration, the first threshold is a minimum daily average lighting duration, and the second threshold is a maximum daily average lighting duration.

In step 608a, when the illumination data or the statistic value of the illumination data is lower than the first threshold, first plant cultivation reference information is generated. The first plant cultivation reference information is configured to recommend enhancing lighting.

In step 608b, when the illumination data or the statistic value of the illumination data is higher than the first threshold and lower than the second threshold, plant cultivation reference information configured to inform that the lighting is suitable, is generated.

In step 608c, when the illumination data or the statistic value of the illumination data is higher than the second threshold, second plant cultivation reference information is generated. The second plant cultivation reference information is configured to recommend reducing lighting.

With the method for generating information provided in the present embodiment, the illumination data is compared with the first threshold and the second threshold in the reference environment data, such that the light-related plant cultivation reference information is generated, thus avoiding stunting the plant because of lacking of lighting or excessive lighting.

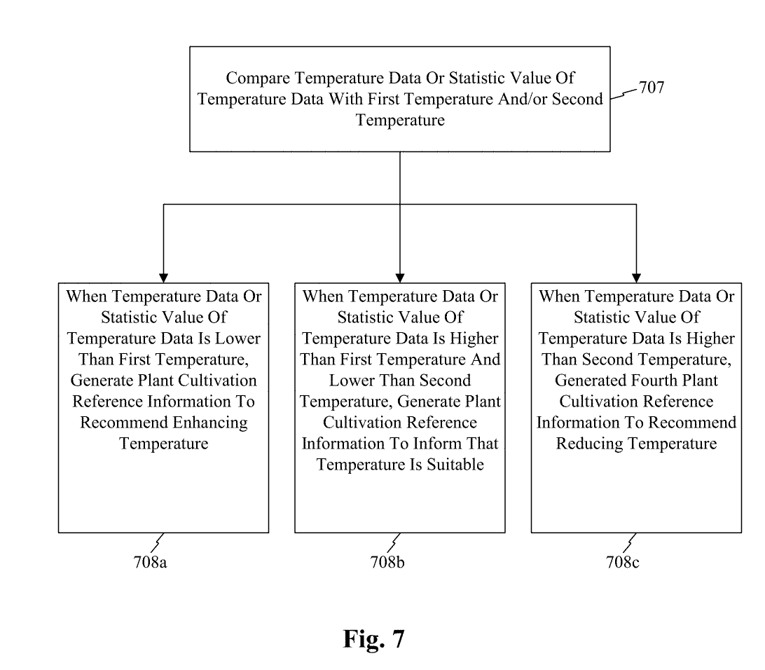

In one embodiment, the growth environment data includes temperature data, and the reference environment data includes a first temperature and a second temperature, in which the first temperature is lower than the second temperature. Accordingly, the following steps in FIG. 7 are performed to implement steps 407 and 408 in FIG. 4.

In step 707, the temperature data or a statistic value of the temperature data is compared with the first temperature, and/or the temperature data or the statistic value of the temperature data is compared with the second temperature.

For example, the temperature data includes the daily average temperature, the first temperature is a minimum daily average temperature, and the second temperature is a maximum daily average temperature.

In step 708a, when the temperature data or the statistic value of the temperature data is lower than the first temperature, third plant cultivation reference information is generated. The third plant cultivation reference information is configured to recommend enhancing temperature.

In step 708b, when the temperature data or the statistic value of the temperature data is higher than the first temperature and lower than the second temperature, plant cultivation reference information configured to inform that the temperature is appropriate is generated.

In step 708c, when the temperature data or the statistic value of the temperature data is higher than the second temperature, fourth plant cultivation reference information is generated. The fourth plant cultivation reference information is configured to recommend reducing temperature.

With the method for generating information provided in the present embodiment, the temperature data is compared with the first temperature and the second temperature in the reference environment data, such that the temperature-related plant cultivation reference information is generated, thereby avoiding stunting the plant because of too low temperature or too high temperature.

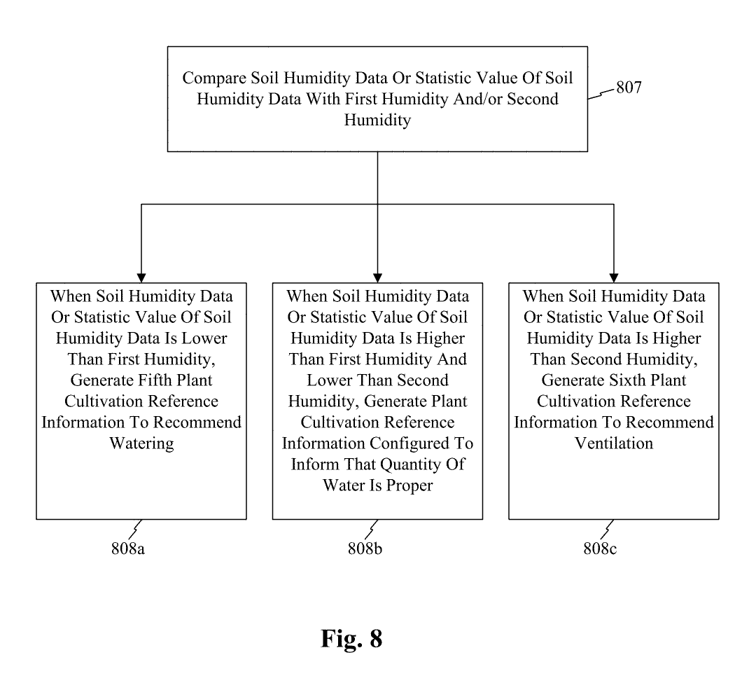

In one embodiment, the growth environment data includes soil humidity data, and the reference environment data includes a first humidity and a second humidity, in which the first humidity is smaller than the second humidity. Accordingly, the following steps in FIG. 8 are performed to implement steps 407 and 408 in FIG. 4.

In step 807, the soil humidity data or a statistic value of the soil humidity data is compared with the first humidity, and/or the soil humidity data or the statistic value of the soil humidity data is compared with the second humidity.

For example, the soil humidity data includes the daily average humidity, the first humidity is a minimum daily average humidity, and the second humidity is a maximum daily average humidity.

In step 808a, when the soil humidity data or a statistic value of the soil humidity data is lower than the first humidity, fifth plant cultivation reference information is generated. The fifth plant cultivation reference information is configured to recommend watering.

In step 808b, when the soil humidity data or a statistic value of the soil humidity data is higher than the first humidity and lower than the second humidity, plant cultivation reference information configured to inform that the quantity of water is suitable is generated.

In step 808c, when the soil humidity data or the statistic value of the soil humidity data is higher than the second humidity, sixth plant cultivation reference information is generated. The sixth plant cultivation reference information is configured to recommend ventilation.

With the method for generating information provided in the present embodiment, the soil humidity data is compared with the first humidity and the second humidity in the reference environment data, such that the moisture-related plant cultivation reference information is generated, thereby avoiding stunting the plant because of too litter water or too much water.

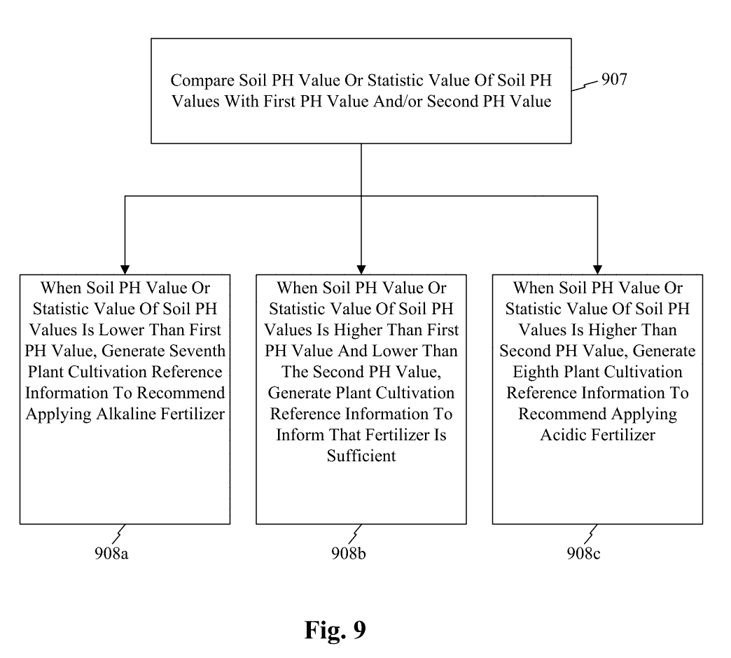

In one embodiment, the growth environment data includes a soil PH value, and the reference environment data includes a first PH value and a second PH value, in which the first PH value is less than the second PH value. Accordingly, the following steps in FIG. 9 are performed to implement steps 407 and 408 in FIG. 4.

In step 907, the soil PH value or a statistic value of the soil PH values is compared with the first PH value, and/or the soil PH value or the statistic value of the soil PH values is compared with the second PH value.

The soil PH value is converted from the soil conductivity. For example, the soil PH value includes a daily average PH value, the first PH value is an acidic PH threshold related to the plant, and the second PH value is an alkali PH threshold related to the plant.

In step 908a, when the soil PH value or the statistic value of the soil PH values is lower than the first PH value, seventh plant cultivation reference information is generated. The seventh plant cultivation reference information is configured to recommend applying an alkaline fertilizer.

In step 908b, when the soil PH value or the statistic value of the soil PH values is higher than the first PH value and lower than the second PH value, plant cultivation reference information configured to inform that fertilizer is suitable is generated.

In step 908c, when the soil PH value or the statistic value of the soil PH values is higher than the second PH value, eighth plant cultivation reference information is generated. The eighth plant cultivation reference information is configured to recommend applying an acidic fertilizer.

With the method for generating information provided in the present embodiment, the soil PH value is compared with the first PH value and the second PH value in the reference environment data, such that the plant cultivation reference information related to the acidity and alkaline fertilizer can be generated, thereby avoiding stunting the plant because some kind of fertilizer is too little or too much.

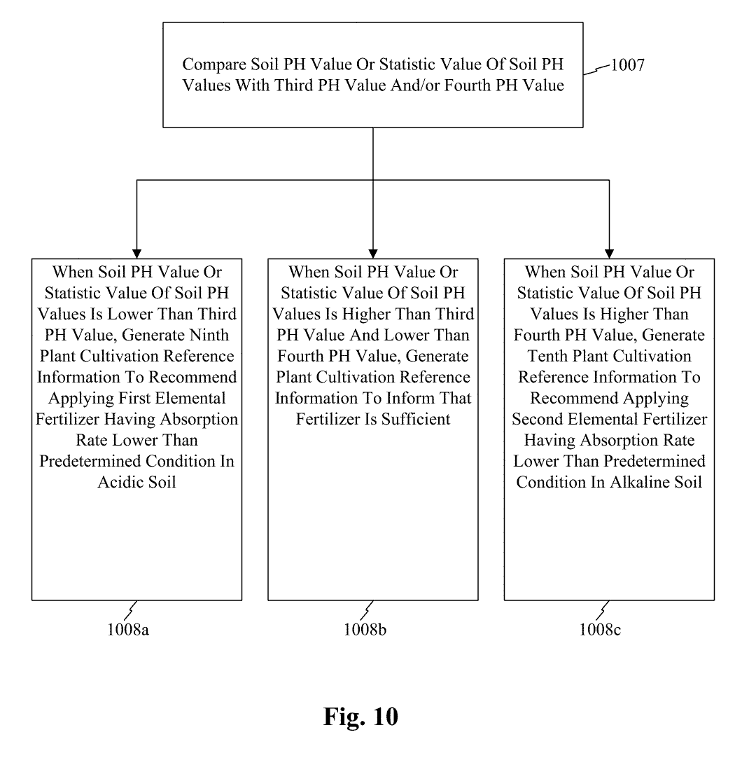

In one embodiment, the growth environment data includes a soil PH value, and the reference environment data includes a third PH value and a fourth PH value, in which the third PH value is lower than the fourth PH value. Accordingly, the following steps in FIG. 10 are performed to implement steps 407 and 408 in FIG. 4.

In step 1007, the soil PH value or a statistic value of the soil PH values is compared with the third PH value, and/or the soil PH value or the statistic value of the soil PH values is compared with the fourth PH value.

The soil PH value is converted from the soil conductivity. For example, the soil PH value includes the daily average PH value, the third PH value is an acidic PH threshold related to the plant, and the fourth PH value is an alkali PH threshold related to the plant.

In step 1008a, when the soil PH value or the statistic value of the soil PH values is lower than the third PH value, ninth plant cultivation reference information is generated. The ninth plant cultivation reference information is configured to recommend applying first elemental fertilizer, and the first elemental fertilizer has an absorption rate lower than a predetermined condition in acidic soil.

For example, the plant in the acidic soil cannot absorb N, P, K and other nutrients well for normal growth, and therefore the user is recommended to apply a fertilizer rich with N, P, K and other nutrients.

In step 1008b, when the soil PH value or the statistic value of the soil PH values is higher than the third PH value and lower than the fourth PH value, plant cultivation reference information configured to inform that fertilizer is suitable is generated.

In step 1008c, when the soil PH value or the statistic value of the soil PH values is higher than the fourth PH value, tenth plant cultivation reference information is generated. The tenth plant cultivation reference information is configured to recommend applying a second elemental fertilizer, and the second elemental fertilizer has an absorption rate lower than a predetermined condition in alkaline soil.

With the method for generating information provided in the present embodiment, the soil PH value is compared with the third PH value and the fourth PH value in the reference environment data, such that the plant cultivation reference information related to nutritional element fertilizer can be generated, thereby avoiding stunting the plant because some kind of fertilizer is too little or too much.

It is to be noted that the above-described embodiments can be implemented in combination with each other, and the present disclosure are not limited thereto.

It is to be noted that the steps performed by the plant monitor in each of the above-described method embodiments can be realized individually as methods for pushing information on the side of the plant monitor; the steps performed by the mobile terminal in each of the above-described method embodiments can be implemented individually as methods for displaying information on the side of the mobile terminal.

With respect to the apparatus in the following embodiment, the specific manners in which each module performs the operation has been described in detail in the embodiments relating to the methods, and will not be repeated herein.

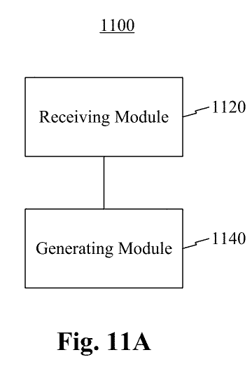

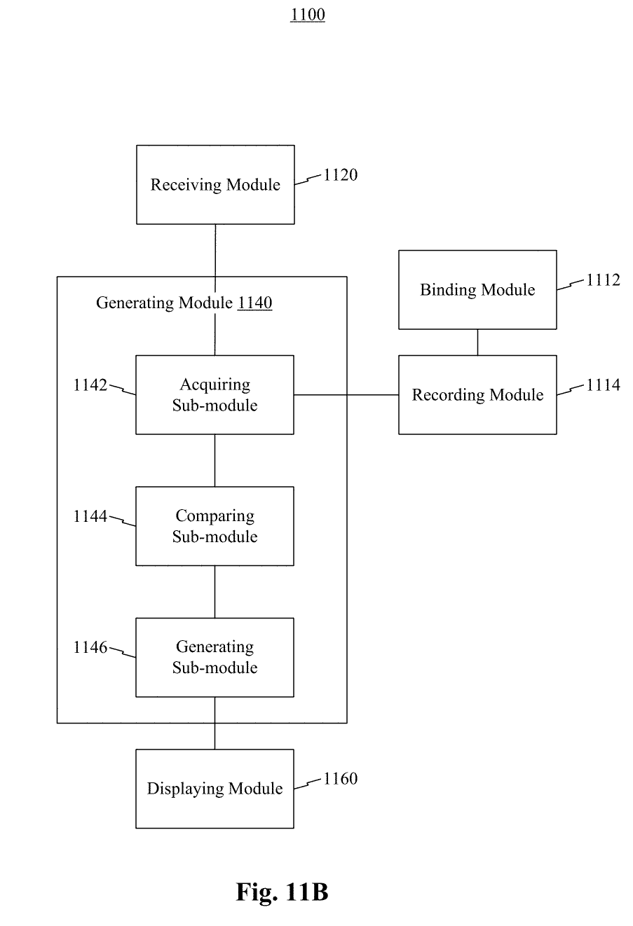

FIG. 11A is a block diagram illustrating an apparatus 1100 for generating information according to an exemplary embodiment of the present disclosure. The apparatus 1100 for generating information can be implemented as a whole or a part of a mobile terminal by a dedicated hardware circuit or a combination of hardware and software. The apparatus 1100 includes: a receiving module 1120 and a generating module 1140.

The receiving module 1120 is configured to receive growth environment data sent by a plant monitor. The growth environment data is collected by the plant monitor via a growth environment monitoring sensor. The growth environment monitoring sensor includes at least one of an illumination sensor, a temperature sensor, a humidity sensor, or a soil conductivity sensor.

The generating module 1140 is configured to generate plant cultivation reference information according to the growth environment data.

With the apparatus 1100 for generating information provided in the present embodiment, the growth environment data of the plant is collected by the at least one growth environment monitoring sensor in the plant monitor, and the growth environment data is sent to the mobile terminal via a wire communication chip, such that the mobile terminal generates the plant cultivation reference information according to the growth environment data. Therefore, the problems that it is difficult for the user to cultivate the ornamental plant to the better state without knowing the living habits may be solved. Moreover, the user only needs to insert the plant monitor into soil to acquire the plant cultivation reference information, without having to know the plant's living habits or having to determine appropriateness of the plant growth environment manually.

In one embodiment, referring to FIG. 11B, the generating module 1140 includes an acquiring sub-module 1142, a comparing sub-module 1144, and a generating sub-module 1146.

The acquiring sub-module 1142 is configured to acquire reference environment data corresponding to a predetermined plant.

The comparing sub-module 1144 is configured to compare the growth environment data with the reference environment data.

The generating sub-module 1146 is configured to generate the plant cultivation reference information according to a comparing result.

In one embodiment, the growth environment data includes illumination data, and the reference environment data includes a first threshold and a second threshold, in which the first threshold is smaller than the second threshold.

The generating sub-module 1146 is configured to: generate first plant cultivation reference information when the illumination data or a statistic value of the illumination data is lower than the first threshold, in which the first plant cultivation reference information is configured to recommend enhancing lighting; and generate second plant cultivation reference information when the illumination data or the statistic value of the illumination data is higher than the second threshold, in which the second plant cultivation reference information is configured to recommend reducing lighting.

In one embodiment, the growth environment data includes temperature data, and the reference environment data includes a first temperature and a second temperature, in which the first temperature is lower than the second temperature.

The generating sub-module 1146 is configured to: generate third plant cultivation reference information when the temperature data or a statistic value of the temperature data is lower than the first temperature, in which the third plant cultivation reference information is configured to recommend enhancing temperature; and generate fourth plant cultivation reference information when the temperature data or the statistic value of the temperature data is higher than the second temperature, in which the fourth plant cultivation reference information is configured to recommend reducing temperature.

In one embodiment, the growth environment data includes soil humidity data, and the reference environment data includes a first humidity and a second humidity, in which the first humidity is smaller than the second humidity.

The generating sub-module 1146 is configured to: generate fifth plant cultivation reference information when the soil humidity data or a statistic value of the soil humidity data is lower than the first humidity, in which the fifth plant cultivation reference information is configured to recommend watering; and generate sixth plant cultivation reference information when the soil humidity data or the statistic value of the soil humidity data is higher than the second humidity, in which the sixth plant cultivation reference information is configured to recommend ventilation.

In one embodiment, the growth environment data includes a soil PH value, and the soil PH value is acquired by calculating soil conductivity, and the reference environment data includes a first PH value and a second PH value, in which the first PH value is less than the second PH value.

The generating sub-module 1146 is configured to: generate seventh plant cultivation reference information when the soil PH value or a statistic value of the soil PH values is lower than the first PH value, in which the seventh plant cultivation reference information is configured to recommend applying an alkaline fertilizer; and generate eighth plant cultivation reference information when the soil PH value or the statistic value of the soil PH values is higher than the second PH value, in which the eighth plant cultivation reference information is configured to recommend applying an acidic fertilizer.

In one embodiment, the growth environment data includes a soil PH value, and the soil PH value is acquired by calculating according to the soil conductivity, and the reference environment data includes a third PH value and a fourth PH value, in which the third PH value is less than the fourth PH value.

The generating sub-module 1146 is configured to: generate ninth plant cultivation reference information when the soil PH value or a statistic value of the soil PH values is lower than the third PH value, in which the ninth plant cultivation reference information is configured to recommend applying a first elemental fertilizer having an absorption rate lower than a predetermined condition in acidic soil; and generate tenth plant cultivation reference information when the soil PH value or the statistic value of the soil PH values is higher than the fourth PH value, in which the tenth plant cultivation reference information is configured to recommend applying a second elemental fertilizer having an absorption rate lower than a predetermined condition in alkaline soil.

In one embodiment, referring to FIG. 11B, the apparatus 1100 further includes a binding module 1112, and a recording module 1114.

The binding module 1112 is configured to establish a binding relationship with the plant monitor.

The recording module 1114 is configured to record the predetermined plant corresponding to the plant monitor.

In one embodiment, referring to FIG. 11B, the apparatus 1100 further includes a displaying module 1160.

The displaying module 1160 is configured to display the plant cultivation reference information and a guidance entrance on a graphical user interface. The guidance entrance includes at least one of an entrance for buying a cultivation implement, an entrance for buying fertilizer, an entrance for jumping to a cultivation forum, or an entrance for jumping to a cultivation technique.

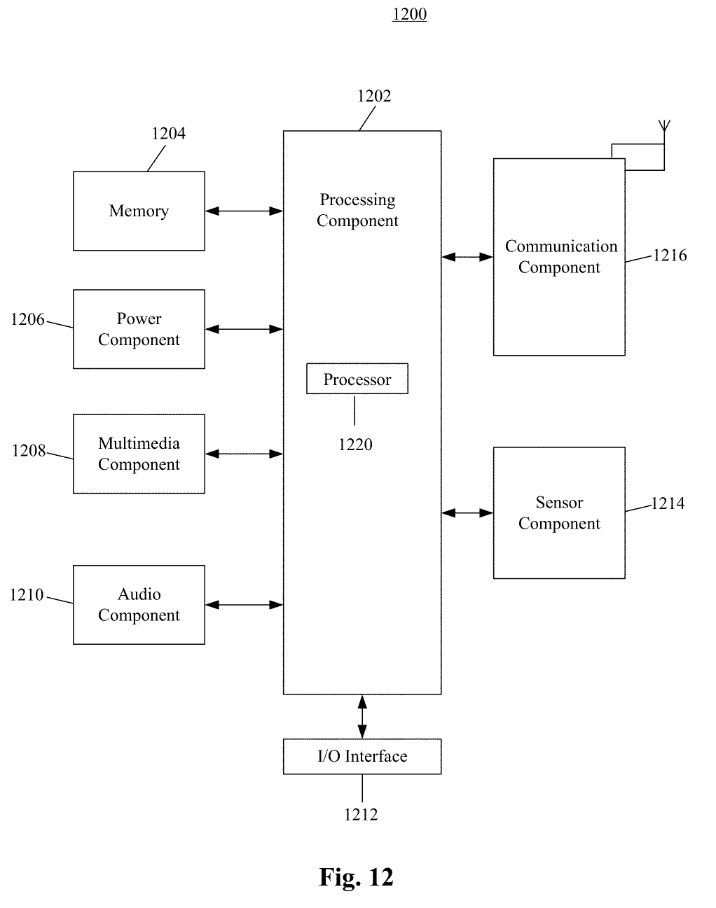

FIG. 12 is a block diagram illustrating a mobile terminal 1200 according to an exemplary embodiment of the present disclosure. For example, the mobile terminal 1200 can be a mobile phone, a computer, a digital broadcasting terminal, a messaging device, a game console, a tablet device, a medical device, fitness equipment, a Personal Digital Assistant PDA, and the like

Referring to FIG. 12, the mobile terminal 1200 can include the following one or more components: a processing component 1202, a memory 1204, a power component 1206, a multimedia component 1208, an audio component 1210, an Input/Output (I/O) interface 1212, a sensor component 1214, and a communication component 1216.

The processing component 1202 typically controls overall operations of the mobile terminal 1200, such as the operations associated with display, telephone calls, data communications, camera operations, and recording operations. The processing component 1202 can include one or more processors 1220 to execute instructions to perform all or part of the steps in the above described methods. Moreover, the processing component 1202 can include one or more modules which facilitate the interaction between the processing component 1202 and other components. For instance, the processing component 1202 can include a multimedia module to facilitate the interaction between the multimedia component 1208 and the processing component 1202.

The memory 1204 is configured to store various types of data to support the operation of the mobile terminal 1200. Examples of such data include instructions for any applications or methods operated on the mobile terminal 1200, contact data, phonebook data, messages, pictures, video, etc. The memory 1204 can be implemented using any type of volatile or non-volatile memory devices, or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The power component 1206 provides power to various components of the mobile terminal 1200. The power component 1206 can include a power management system, one or more power sources, and any other components associated with the generation, management, and distribution of power in the mobile terminal 1200.

The multimedia component 1208 includes a screen providing an output interface between the mobile terminal 1200 and the user. In some embodiments, the screen can include a liquid crystal display (LCD) and a press panel (TP). If the screen includes the press panel, the screen can be implemented as a press screen to receive input signals from the user. The press panel includes one or more press sensors to sense presses, swipes, and other gestures on the press panel. The press sensors can not only sense a boundary of a press or swipe action, but also sense a duration time and a pressure associated with the press or swipe action. In some embodiments, the multimedia component 1208 includes a front camera and/or a rear camera. The front camera and/or the rear camera can receive external multimedia data while the mobile terminal 1200 is in an operation mode, such as a photographing mode or a video mode. Each of the front camera and the rear camera can be a fixed optical lens system or have focus and optical zoom capability.

The audio component 1210 is configured to output and/or input audio signals. For example, the audio component 1210 includes a microphone (MIC) configured to receive an external audio signal when the mobile terminal 1200 is in an operation mode, such as a call mode, a recording mode, and a voice recognition mode. The received audio signal can be further stored in the memory 1204 or transmitted via the communication component 1216. In some embodiments, the audio component 1210 further includes a speaker to output audio signals.

The I/O interface 1212 provides an interface for the processing component 1202 and peripheral interface modules, such as a keyboard, a click wheel, buttons, and the like. The buttons can include, but are not limited to, a home button, a volume button, a starting button, and a locking button.

The sensor component 1214 includes one or more sensors to provide status assessments of various aspects of the mobile terminal 1200. For instance, the sensor component 1214 can detect an open/closed status of the mobile terminal 1200 and relative positioning of components (e.g. the display and the keypad of the mobile terminal 1200. The sensor component 514 can also detect a change in position of the mobile terminal 1200 or of a component in the mobile terminal 1200, a presence or absence of user contact with the mobile terminal 1200, an orientation or an acceleration/deceleration of the mobile terminal 1200, and a change in temperature of the mobile terminal 1200. The sensor component 1214 can include a proximity sensor configured to detect the presence of nearby objects without any physical contact. The sensor component 1214 can also include a illumination sensor, such as a CMOS or CCD image sensor, for use in imaging applications. In some embodiments, the sensor component 1214 can also include an accelerometer sensor, a gyroscope sensor, a magnetic sensor, a pressure sensor, or a temperature sensor.

The communication component 1216 is configured to facilitate wired or wireless communication between the mobile terminal 1200 and other devices. The mobile terminal 1200 can access a wireless network based on a communication standard, such as WIFI, 2G, 3G, or 4G, or a combination thereof. In one exemplary embodiment, the communication component 1216 receives a broadcast signal or broadcast associated information from an external broadcast management system via a broadcast channel. In one exemplary embodiment, the communication component 1216 further includes a near field communication (NFC) module to facilitate short-range communications. For example, the NFC module can be implemented based on a radio frequency identification (RFID) technology, an infrared data association (IrDA) technology, an ultra-wideband (UWB) technology, a Bluetooth (BT) technology, and other technologies.

In exemplary embodiments, the mobile terminal 1200 can be implemented with one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing the above described methods.

In exemplary embodiments, there is also provided a non-transitory computer readable storage medium including instructions, such as included in the memory 1204. The instructions can be performed by the processor 1220 of the mobile terminal 1200 to perform the above described methods for generating information. For example, the non-transitory computer-readable storage medium can be a ROM, a RAM, a CD-ROM, a magnetic tape, a floppy disc, an optical data storage device, and the like.

One of ordinary skill in the art will understand that the above described modules can each be implemented by hardware, or software, or a combination of hardware and software. One of ordinary skill in the art will also understand that multiple ones of the above described modules may be combined as one module, and each of the above described modules may be further divided into a plurality of sub-modules.

Other embodiments of the disclosure will be apparent to those skilled in the art from consideration of the specification and practice of the disclosure disclosed here. This application is intended to cover any variations, uses, or adaptations of the disclosure following the general principles thereof and including such departures from the present disclosure as come within known or customary practice in the art. It is intended that the specification and examples be considered as exemplary only, with a true scope and spirit of the disclosure being indicated by the following claims.

It will be appreciated that the present disclosure is not limited to the exact construction that has been described above and illustrated in the accompanying drawings, and that various modifications and changes can be made without departing form the scope thereof. It is intended that the scope of the disclosure only be limited by the appended claims.

What is claimed is:

1. A plant monitor, comprising:
a main control chip;
a wireless communication chip connected with the main control chip; and
at least one growth environment monitoring sensor connected with the main control chip,
wherein
the at least one growth environment monitoring sensor comprises at least one of an illumination sensor, a temperature sensor, a humidity sensor, or a soil conductivity sensor, and
the main control chip is configured to acquire growth environment data collected by the at least one growth environment monitoring sensor, and send the growth environment data to a mobile terminal via the wireless communication chip, such that the mobile terminal generates plant cultivation reference information according to the growth environment data.

2. The plant monitor according to claim 1, further comprising:
an insertion arm configured to be inserted into soil,
wherein the humidity sensor is disposed on the insertion arm, and configured to collect soil humidity.

3. The plant monitor according to claim 1, further comprising:
an insertion structure configured to be inserted into soil,
wherein the soil conductivity sensor comprises two metal electrodes, and the two metal electrodes are disposed on the insertion structure.

4. The plant monitor according to claim 3, wherein the insertion structure comprises:
two insertion arms disposed in parallel, each insertion arm being provided with one metal electrode.

5. The plant monitor according to claim 1, wherein:
the main control chip is further configured to establish a binding relationship with the mobile terminal via the wireless communication chip.

6. A method for generating information, comprising:
receiving growth environment data sent by a plant monitor, wherein the growth environment data is collected by the plant monitor via a growth environment monitoring sensor, and the growth environment monitoring sensor comprises at least one of an illumination sensor, a temperature sensor, a humidity sensor, or a soil conductivity sensor; and
generating plant cultivation reference information according to the growth environment data.

7. The method according to claim 6, wherein generating plant cultivation reference information according to the growth environment data comprises:
acquiring reference environment data corresponding to a predetermined plant;
comparing the growth environment data with the reference environment data; and
generating the plant cultivation reference information according to a comparing result.

8. The method according to claim 7, wherein the growth environment data comprises illumination data, and the reference environment data comprises a first threshold and a second threshold, and the first threshold is smaller than the second threshold, and
generating the plant cultivation reference information according to a comparing result comprises:
when the illumination data or a statistic value of the illumination data is lower than the first threshold, generating first plant cultivation reference information, wherein the first plant cultivation reference information is configured to recommend enhancing illumination; and
when the illumination data or the statistic value of the illumination data is higher than the second threshold, generating second plant cultivation reference information, wherein the second plant cultivation reference information is configured to recommend reducing illumination.

9. The method according to claim 7, wherein the growth environment data comprises temperature data, and the reference environment data comprises a first temperature and a second temperature, and the first temperature is lower than the second temperature; and
generating the plant cultivation reference information according to a comparing result comprises:
when the temperature data or a statistic value of the temperature data is lower than the first temperature, generating first plant cultivation reference information, wherein the first plant cultivation reference information is configured to recommend enhancing temperature; and
when the temperature data or the statistic value of the temperature data is higher than the second temperature, generating second plant cultivation reference information, wherein the second plant cultivation reference information is configured to recommend reducing temperature.

10. The method according to claim 7, wherein the growth environment data comprises soil humidity data, and the reference environment data comprises a first humidity and a second humidity, and the first humidity is lower than the second humidity; and
generating the plant cultivation reference information according to a comparing result comprises:
when the soil humidity data or a statistic value of the soil humidity data is lower than the first humidity, generating first plant cultivation reference information, wherein the first plant cultivation reference information is configured to recommend watering; and
when the soil humidity data or the statistic value of the soil humidity data is higher than the second humidity, generating second plant cultivation reference information, wherein the second plant cultivation reference information is configured to recommend ventilation.

11. The method according to claim 7, wherein the growth environment data comprises a soil PH value, and the soil PH value is acquired by calculating according to the soil conductivity, and the reference environment data comprises a first PH value and a second PH value, and the first PH value is lower than the second PH value; and
generating the plant cultivation reference information according to a comparing result comprises:
when the soil PH value or a statistic value of the soil PH values is lower than the first PH value, generating first plant cultivation reference information, wherein the first plant cultivation reference information is configured to recommend applying an alkaline fertilizer; and
when the soil PH value or the statistic value of the soil PH values is higher than the second PH value, generating second plant cultivation reference information, wherein the second plant cultivation reference information is configured to recommend applying an acidic fertilizer.

12. The method according to claim 7, wherein the growth environment data comprises a soil PH value, and the soil PH value is acquired by calculating according to the soil conductivity, and the reference environment data comprises a first PH value and a second PH value, and the first PH value is lower than the second PH value; and
generating the plant cultivation reference information according to a comparing result comprises:
when the soil PH value or a statistic value of the soil PH values is lower than the first PH value, generating first plant cultivation reference information, wherein the first plant cultivation reference information is configured to recommend applying first elemental fertilizer, and the first elemental fertilizer has an absorption rate lower than a predetermined condition in acidic soil; and
when the soil PH value or the statistic value of the soil PH values is higher than the second PH value, generating second plant cultivation reference information, wherein the second plant cultivation reference information is configured to recommend applying second elemental fertilizer, and the second elemental fertilizer has an absorption rate lower than a predetermined condition in alkaline soil.

13. The method according to claim 7, further comprising:
establishing a binding relationship with the plant monitor; and
recording the predetermined plant corresponding to the plant monitor.

14. The method according to claim 6, further comprising:
displaying the plant cultivation reference information and a guidance entrance on a graphical user interface, wherein the guidance entrance comprises at least one of an entrance for buying a cultivation implement, an entrance for buying fertilizer, an entrance for jumping to a cultivation forum, or an entrance for jumping to a cultivation technique.

15. An apparatus for generating information, comprising:
a processor; and
a memory configured to store an instruction executable by the processor;
wherein the processor is configured to:
receive growth environment data sent by a plant monitor, wherein the growth environment data is collected by the plant monitor via a growth environment monitoring sensor, and the growth environment monitoring sensor comprises at least one of an illumination sensor, a temperature sensor, a humidity sensor, or a soil conductivity sensor; and
generate plant cultivation reference information according to the growth environment data.

16. The apparatus according to claim 15, wherein the processor is configured to:
acquire reference environment data corresponding to a predetermined plant;
compare the growth environment data with the reference environment data;
generate the plant cultivation reference information according to a comparing result.

17. The apparatus according to claim 16, wherein the processor is further configured to:
establish a binding relationship with the plant monitor; and
record the predetermined plant corresponding to the plant monitor.

18. The apparatus according to claim 15, further comprising:
a display configured to display the plant cultivation reference information and a guidance entrance on a graphical user interface, wherein the guidance entrance comprises at least one of an entrance for buying a cultivation implement, an entrance for buying fertilizer, an entrance for jumping to a cultivation forum, or an entrance for jumping to a cultivation technique.
